(19) United States
(12) Patent Application Publication
(10) Pub. No.: US 2008/0085876 A1
(43) Pub. Date: Apr. 10, 2008

(54) Insect sting treatment

(76) Inventor: David L. Schwartz, Hanover Township, PA (US)

Correspondence Address: L. ZALE PATENT LAW OFFICE, 334 SOUTH FRANKLIN ST., WILKES-BARRE, PA, 18702, US

(21) Appl. No.: 11/895,747

(22) Filed: Aug. 27, 2007

Related U.S. Application Data
(60) Provisional application No. 60/849,708, filed on Oct. 5, 2006.

(51) Int. Cl. A61K 31/56; A61P 17/02

(52) U.S. Cl. 514/178; 514/169; 514/789

(57) ABSTRACT

A treatment for insect bites and stings [100] includes a mixture of a base material [101], a draw-out agent [102], an immune suppression agent [103] for reducing an immune response, and optionally, an antimicrobial agent [104]. The draw-out agent [102] functions to keep the wound moist and open. The immune suppression agent [103] reduces the body's immune reaction keeping the wound open allowing for ejection of foreign bodies, microbes and toxins. The natural antimicrobial properties of the antimicrobial agent [104] kill the microbes. Optionally, the treatment [100] may include insect venom antidotes [105] to neutralize the venom's effect.

### CROSS REFERENCE TO RELATED APPLICATIONS

The present application claims priority from U.S. Provisional Patent application 60/849,708 filed Oct. 5, 2006 by the same inventor, David Schwartz.

### BACKGROUND OF THE INVENTION

1. Field of the Invention

The present invention relates to a treatment for insect stings, and more specifically a treatment for insect stings which is more effective than prior art treatments.

2. Discussion of Related Art

Insects use their sharp protrusions to penetrate the skin of a person. These may be their proboscis, barbs or stingers which are typically covered with microbes, many of which may be pathogenic.

Therefore after an insect bite or sting, microbes are forced into and under the skin. Typically these microbes cause infection, inflammation, pain and redness.

Some of these insects also embed a portion of the insect inside the wound, such as the stinger. This foreign body usually triggers the body's immune response protecting it against the embedded foreign cells and materials. This immune response is a chain reaction of chemical events which results in inflammation, itching, redness and pain.

When a foreign body is lodged within the body of a person, the reaction persists until the foreign body has been removed.

Insects, may also inject a venom or toxin into the wound. This is typically the case with spider bites. Spiders inject a powerful neurotoxin into the wound which cause significant reactions, especially in victims who are allergic to substances such venom.

And finally, the insects may be infected with other microbes which permeate their bodies, including their saliva. Therefore, when an infected insect bites a person, saliva is introduced into the wound along with the microbes in the saliva. This is how malaria is spread.

Therefore, microbes, foreign body of the insect, venoms, toxins, and secondary microbes of infected insects introduced into the wound must be neutralized or removed from the person's body.

One such treatment used in the past was a local anesthetic, such as benzocaine. This only treated the pain but did not affect infections, the toxins or remove the foreign body.

Another common treatment was to let the wound dry and create a scab. This will, unfortunately, lock in the microbes, foreign bodies, and toxins underneath the skin. The infection is now sealed under the skin and adverse reactions persist for a longer period of time.

People were also treated with steroids to reduce the body's immune reactions. These helped at reducing swelling and itching, but did not address the other problems listed above.

Coal tar or Ichthammol have been used in the past as the draw-out salve for drawing out objects such as slivers which had punctured the skin. These functioned to keep the skin pliable, allowing the sliver to work its way out to the surface. It was mentioned for insect bites and bee stings, but was listed as not as effective as the topical steroids above and therefore not recommended for these.

Currently, there is a need for a substance which reduces the symptoms of insect bites or stings, and which actively aids in the healing of these insect bites and stings.

### SUMMARY OF THE INVENTION

One embodiment of the present invention is a treatment [100] for a puncture [27] in skin of a person or animal, from an insect sting or bite comprising:
a) a thick non-evaporating protective base material [101];
b) a draw-out agent [102] to keep the puncture site from sealing allowing any foreign bodies embedded under the skin to be forced out; and
c) an immune suppression agent [103] for reducing the immune response of said person or animal near the puncture [27].

In an alternative embodiment, the immune suppression agent [103], the base material [101], the draw-out agent and the antimicrobial agent are Ichthammol.

The invention may also be embodied as an improved method for treating a puncture in skin of a person or animal, from an insect sting or bite comprising:
a) applying an immune suppression agent [103] of said person or animal near the puncture;
b) applying a draw-out agent [102] to keep the puncture site from sealing allowing any foreign bodies embedded under the skin to be forced out; and
c) applying a base material [101] which is thick and non-evaporating on said puncture to protect the puncture.

### OBJECTS OF THE INVENTION

It is an object of the present invention to provide a treatment for insect bites and stings which is faster acting than prior art treatments.

It is another object of the present invention to provide a treatment for insect bites and stings which reduces secondary infections.

It is another object of the present invention to provide a treatment for insect bites and stings which reduces swelling.

It is another object of the present invention to provide a treatment for insect bites and stings which reduces the body's natural immune response.

It is another object of the present invention to provide a treatment for insect bites and stings which draws out injected venom or toxin.

### BRIEF DESCRIPTION OF THE DRAWINGS

The advantages of this disclosure will become more apparent when read with the specification and the drawings, wherein:

FIG. 1 illustrates a bee stinger having microbes attached to its surface, approaching a person's skin also having microbes attached to its surface.

FIG. 2 illustrates the bee stinger of FIG. 1 puncturing the person's skin of FIG. 1 creating a wound.

FIG. 3 illustrates a portion of the stinger embedded in the wound created by the stinger.

FIG. 4 illustrates the wound a short time after insertion of the stinger.

FIG. 5 illustrates the wound a short time after insertion of the stinger that has been treated with the mixture according to the present invention.

### DETAILED DESCRIPTION OF THE INVENTION

Ichthammol

Ichthammol is bituminosulfate derived from heating coal. It is a sulfonated hydrocarbon (shale oil), typically brown, having the consistency of tar.

It has a thick and grease-like consistency which does not easily evaporate. Since it is also soluble in water, it is a very good emollient used to moisturize skin and keep the skin from getting dry or crusty for long periods of time.

Antimicrobial

Since Ichthammol has a sulfonic component, it creates a chemical environment making it difficult for microbes to survive. It is efficient at neutralizing gram positive organisms.

Therefore, it is also listed as an antimicrobial agent.

Immune Response

When the body identifies foreign material or cells in its presence, the human body triggers a long chain of chemical reactions which result in itching, swelling, pain and redness.

There are intermediate substances produced by these reactions which continue and are processed ultimately into final products.

Steroids

Chemicals, such as hydrocortisone and other steroids modulate these chain reactions by modifying the intermediate substances making them non-reactive, and thereby slowing or stopping the immune response. Therefore, this reduces itching, swelling, redness and pain.

It has been found that use of an emollient, antimicrobial, draw-out salve and a steroid as a topical preparation applied and protected with a bandage causes significantly increased efficacy.

Applicants have set forth their beliefs as to the proposed mechanism to explain this unexpected enhancement of the effectiveness of the present invention.

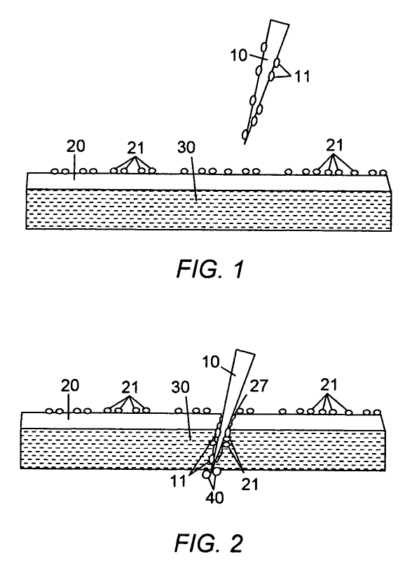

FIG. 1 illustrates bee stinger 10 having microbes 11 attached to the surface of the stinger 10, approaching a person's skin 20 also having microbes 21 attached to its surface.

FIG. 2 illustrates the bee stinger 10 puncturing the person's skin 20 creating a wound 27 and inserting the microbes 21 on the surface of the skin 20, the stinger 10 with its microbes 11 and toxins 40 into the wound 27.

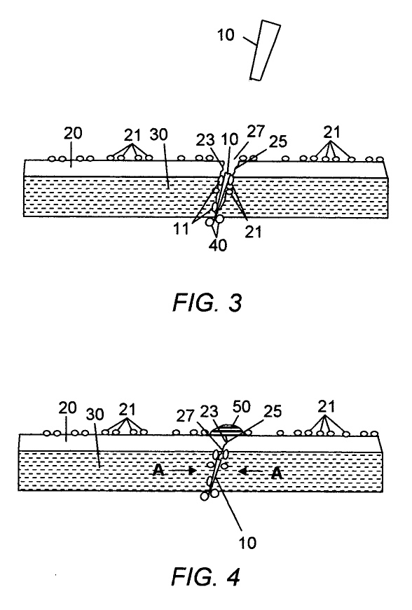

FIG. 3 illustrates a portion of the stinger 10 embedded in wound 27 along with the injected skin microbes 21 and stinger microbes 11 and toxins 40. Would edges 23 and 25 can be seen here.

FIG. 4 illustrates the wound 27 a short time after insertion of the stinger 10. The immune response causes swelling of the nearby tissue 30. Swelling causes wound edge 23 to move toward wound edge 25 thereby closing up wound 27 and causing wound edges 23, 25 to move toward each other in the direction of the arrows marked “A”. Also fibrinogen begins to create fibrins to create a scab 50 which fills and seals wound 27. The stinger 10 cannot easily move backward out of wound 27 since it is now sealed. The immune reaction and infection will continue for a longer period of time when stinger is in wound 27.

Also, skin microbes 21 and stinger microbes 11, and stinger toxin 40 are all sealed inside, under skin 20. These will cause infections and adverse reactions until they are neutralized or removed. Again, it is difficult for them to be expelled since wound 27 is sealed.

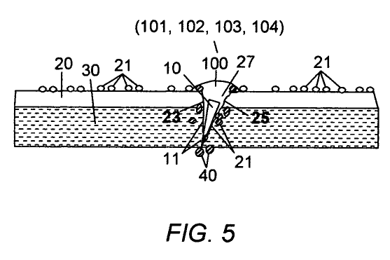

FIG. 5 is an enlarged view of the wound 27 a short time after insertion of the stinger 10 that has been treated with the treatment 100 of the present invention.

Mixture 100 in one embodiment employs a base agent 101 for protecting a wound, a draw-out agent 102 for drawing foreign bodies out of tissue, which may be an emollient, an immune response suppression agent 103 for reducing an immune response near a wound, and optionally an antimicrobial agent 104, and/or an insect venom antidote 105. These may be selected from known substances which have the same function.

Mixture 100 is applied prior to closure of wound 27 and formation of scab 50. It is allowed to cover the immediate surface near wound 27 and pass into wound 27 between wound edges 23, 25. The emollient properties of the draw-out agent 102 in mixture 100 keep the tissues moist reducing or preventing creation of the scab 50, thereby keeping wound 27 open. This allows the tissue around the wound to work foreign bodies out of the tissue.

The steroids stop the closing of wound 27 by reducing swelling of tissues 30. This now leaves an open path for backing the stinger 10 out of wound 27.

Also, the antimicrobial properties of mixture 100 kill the microbes 11,21 in wound 27.

The tar-like texture of Ichthammol may be used as base material 101 which creates a protective cap sealing off wound 27, while still allowing stinger 10 to pass through it as it is pushed out of wound 27.

Other tar-based materials may also be used as base material 101 to provide a protective covering.

Ichthammol also has emollient and antimicrobial properties and therefore may be used as the draw out agent 102 and antimicrobial agent 104.

In optional embodiments, other currently known base materials 101, draw out agents 102 and antimicrobial agents 104 may be used.

Also, the most effective immune suppression agent 103 is hydrocortisone. However, this invention will cover other known immune suppression agents, such as other variations of cortisone and other topical steroids which reduce or suspend the body's immune response.

The most effective mixture was a 50% mix by weight of an Ichthammol preparation (having 20% active ingredient by weight), with 50% by weight of a hydrocortisone preparation (having 1% active ingredient by weight).

In another optional embodiment, the treatment according to the present invention may include a known spider venom antidote. This dose should be that described by the manufacturer.

Since other modifications and changes varied to fit particular operating requirements and environments will be apparent to those skilled in the art, the invention is not considered limited to the example chosen for the purposes of disclosure, and covers all changes and modifications which do not constitute departures from the true spirit and scope of this invention.

What is claimed is:

1. A treatment [100] for a puncture [27] in skin of a person or animal, from an insect sting or bite comprising:
a) a thick non-evaporating protective base material [101];
b) a draw-out agent [102] to keep the puncture site from sealing allowing any foreign bodies embedded under the skin to be forced out; and
c) an immune suppression agent [103] for reducing the immune response of said person or animal near the puncture [27].

2. The treatment [100] of claim 1 further comprising:
an antimicrobial agent [104].

3. The treatment [100] of claim 1 wherein the immune suppression agent [103] also has anti-inflammatory properties.

4. The treatment [100] of claim 1 wherein the immune suppression agent [103] is a topical steroid.

5. The treatment [100] of claim 1 wherein the immune suppression agent [103] is of the cortisone family.

6. The treatment [100] of claim 1 wherein the immune suppression agent [103] is hydrocortizone.

7. The treatment [100] of claim 1 wherein the base material is a tar-based material.

8. The treatment [100] of claim 1 wherein the base material is Ichthammol.

9. The treatment [100] of claim 1 wherein the draw-out agent is Ichthammol.

10. The treatment [100] of claim 1 wherein the antimicrobial agent is Ichthammol.

11. The treatment [100] of claim 1 further comprising:
an insect venom antidote.

12. The treatment [100] of claim 1 further comprising: a spider venom antidote.

13. An improved method for treating a puncture in skin of a person or animal, from an insect sting or bite comprising:
a) applying an immune suppression agent [103] of said person or animal near the puncture;
b) applying a draw-out agent [102] to keep the puncture site from sealing allowing any foreign bodies embedded under the skin to be forced out; and
c) applying a base material [101] which is thick and non-evaporating on said puncture to protect the puncture.

14. The improved method of claim 13 further comprising the step of:
applying an antimicrobial agent [104] to said puncture after applying the immune suppression agent [103].

15. The improved method of claim 13 further comprising the step of:
applying an agent having anti-inflammatory properties.

16. The improved method of claim 13 wherein the step of applying an immune suppression agent [103] comprises the step of:
applying a topical steroid.

17. The improved method of claim 13 wherein the step of applying an immune suppression agent [103] comprises the step of:
applying an agent from the cortisone family.

18. The improved method of claim 13 wherein the step of applying a base material [101] comprises the step of:
applying Ichthammol.

19. The improved method of claim 13 wherein the draw-out agent [102] is Ichthammol.

20. The treatment of claim 13 further comprising:
an insect venom antidote.
